(19) United States
(12) Patent Application Publication
(10) Pub. No.: US 2023/0317244 A1
(43) Pub. Date: Oct. 5, 2023

(54) SYSTEMS AND METHODS FOR A TREATMENT AND SOCIAL MEDIA PLATFORM

(71) Applicant: SUN Behavioral Health, Inc., Red Bank, NJ (US)

(72) Inventors: Steven Stuyvesant Page, Fair Haven, NJ (US); Christopher James Jagoditz, Franklin, TN (US); E. Daniel Thomas, JR., Louisville, KY (US); Thomas E. Rourke, Plano, TX (US); Lance Wayne Folske, Dallas, TX (US)

(21) Appl. No.: 17/655,844

(22) Filed: Mar. 22, 2022

(51) Int. Cl. G06Q 50/00; G06Q 10/10; G16H 20/70

(52) U.S. Cl. CPC G16H 20/70; G06Q 10/1097; G06Q 50/01

(57) ABSTRACT

A system for a treatment and social media platform may include a processor that, in response to executing executable instructions, may be operable to: obtain, at a computing platform, a first data packet from a user device; generate, on the computing platform, a task data object based on the first data packet, where the task data object may include text data or a data field for a completion date; generate, on the computing platform, a first social media post data object that may include some of the text data or may correspond to a first social media post on the computing platform; obtain, at the computing platform, a second data packet from the user device that may include a completion date; and update the task data object by including, in the data field, the completion date of the second data packet.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### BACKGROUND OF THE DISCLOSURE

The present disclosure generally relates to social media platforms, and more particularly to systems and methods for a treatment and social media platform.

Social media platforms are technological solutions that allow people to connect with others that they may not otherwise be able to do so in person. For example, a person may want to connect and share information with others, but may not be able to do so in person because the person is geographically isolated or the person’s interests are so specialized that he or she cannot find others with the same specialized interests. While these social media platforms help digitally connect people, they have several technological limitations. For example, social media platforms do not have task or activity tracking capabilities. Furthermore, social media platforms often require or encourage users to use the users’ real information (name, location, etc.) when using an account on the platform, which prevents users from remaining anonymous.

In treating mental and emotional problems, a patient may set goals, and achieving those goals may increase the effectiveness of the treatment. When other people know about these goals and are able to encourage accountability with the patient, or when other people know that the patient has achieved a goal, the effectiveness of the treatment may increase at an even higher rate. However, the patient is often limited by how other people can encourage him or her because, for example, the patient may have limited in-person connections or may wish to remain anonymous during treatment. Thus, certain benefits provided by social media platforms are not available to such patients, in addition to the technology limitations of social media platforms.

What is needed then are systems and methods for a treatment and social media platform.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a system. The system may include a system for a treatment and social media platform. The system may include a processor. The system may include a computer-readable storage medium with executable instructions stored therein. The processor, in response to executing the executable instructions, may be operable to perform one or more steps. The steps may include obtaining, at a computing platform, a first data packet from a user device associated with a first user account of the computing platform. The first data packet may include text data. The steps may include generating, on the computing platform, a task data object based on the first data packet. The task data object may include at least a portion of the text data of the first data packet or a data field for a completion date. The steps may include generating, on the computing platform, a first social media post data object. The first social media post data object may include at least a portion of the text data of the first data packet. The first social media post data object may correspond to a first social media post on the computing platform that may be viewable by one or more users of the computing platform. The steps may include obtaining, at the computing platform, a second data packet from the user device associated with the first user account. The second data packet may include a completion date. The steps may include updating the task data object by including, in the data field, the completion date of the second data packet.

Another aspect of the disclosure may include a computer-implemented method. The computer-implemented method may include a computer-implemented method for a treatment and social media platform. The computer-implemented method may include one or more of the steps carried out by the processor disclosed above.

Another aspect of the disclosure is another system. The system may include a system for a treatment and social media platform. The system may include a processor. The system may include a computer-readable storage medium with executable instructions stored therein. The processor, in response to executing the executable instructions, may be operable to perform one or more steps. The steps may include obtaining, at a treatment platform, a first data packet from a user device associated with a user account of the treatment platform. The first data packet may include text data. The steps may include generating, on the treatment platform, a task data object based on the first data packet. The task data object may include a data field for a completion date. The steps may include sending, to a social media platform, a first message based on the task data object. The first message may include at least a portion the text data of the first data packet. At least a portion of the text data may be viewable by one or more users of the social media platform. The steps may include obtaining, at the treatment platform, a second data packet from the user device associated with the user account. The second data packet may include a completion date. The steps may include updating the task data object by including, in the data field, the completion date of the second data packet. The steps may include sending, to the social media platform, a second message based on the updated task data object. The second message may include the completion date. The completion date may be viewable by one or more users of the social media platform.

The systems and methods disclosed herein provide technological solutions to technological problems in the field of social media platforms. For example, by having a first user (e.g., a patient undergoing treatment) create a first data packet via his/her user device with goal information and data and sending the first data packet to the computing platform, the computing platform can generate a task data object, a social media post data object, and a corresponding social media post. The social media post can be viewable by one or more users of the computing platform. In this way, the computing platform can automatically use goal data to create a social media post to help the patient receive encouragement from other users of the platform, which increases the effectiveness of the patient’s treatment. Additionally, by having the first user create a second data packet with data indicating completion of the goal, the computing platform can update the task data object and either update the social media post or create a new social media post automatically. This further allows other users to encourage the patient in increases the effectiveness of treatment. Furthermore, such functionality improves the social media platform aspect of the system by combining goal tracking feature with automated social media features. Furthermore, such systems and methods effect a particular treatment or prophylaxis for a medical condition because such specific steps increase the effectiveness of treatment and have more than a nominal or insignificant relationship to the treatment.

Numerous other objects, advantages and features of the present disclosure will be readily apparent to those of skill in the art upon a review of the following drawings and description of various embodiments.

### BRIEF DESCRIPTION OF THE DRAWINGS

FIG. 1 is a schematic block diagram illustrating one embodiment of a system for a treatment and social media platform.

FIG. 2A is a flowchart diagram illustrating one embodiment of a computer-implemented method for a treatment and social media platform.

FIG. 2B is a flowchart diagram continuing the embodiment of the computer-implemented method of FIG. 2A.

FIG. 3 is a front view illustrating one embodiment of a user interface displayable on a user device related to a system for a treatment and social media platform.

FIG. 4 is a front view illustrating one embodiment of a user interface displayable on a user device related to a system for a treatment and social media platform.

FIG. 5 is a front view illustrating one embodiment of a user interface displayable on a user device related to a system for a treatment and social media platform.

FIG. 6 is a front view illustrating one embodiment of a user interface displayable on a user device related to a system for a treatment and social media platform.

FIG. 7 is a schematic block diagram illustrating one embodiment of a system for a treatment and social media platform.

FIG. 8A is a flowchart diagram illustrating one embodiment of a computer-implemented method for a treatment and social media platform.

FIG. 8B is a flowchart diagram continuing the embodiment of the computer-implemented method of FIG. 8A.

### DETAILED DESCRIPTION

While the making and using of various embodiments of the present disclosure are discussed in detail below, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatus and methods described herein. Such equivalents are considered to be within the scope of this disclosure and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

As used herein, the term “computing device” may include a desktop computer, a laptop computer, a tablet computer, a mobile device such as a mobile phone or a smart phone, a smartwatch, a gaming console, an application server, a database server, or some other type of computing device. A computing device may include a physical computing device or may include a virtual machine (VM) executing on another computing device. A computing device may include a cloud computing system, a distributed computing system, or another type of multi-device system.

As used herein, the term “data network” may include a local area network (LAN), wide area network (WAN), the Internet, or some other network. A data network may include one or more routers, switches, repeaters, hubs, cables, or other data communication components. A data network may include a wired connection or a wireless connection.

As used herein, the term “computing platform” or “platform” may include a computing environment where a portion of software can execute. A computing platform may include hardware on which the software may execute. The computing platform may include an operating system. The computing platform may include one or more software applications, scripts, functions, or other software. The computing platform may include one or more application programming interfaces (APIs) by which different portions of the software of the platform may communicate with each other or invoke functions. The computing platform may include one or more APIs by which it may communicate with external software applications or by which external software applications may interact with the platform. The computing platform may include a software framework. The computing platform may include one or more VMs. The software platform may include one or more data storages. The software platform may include a client application that executes on an external computing device and that interacts with the platform in a client-server architecture.

As used herein, the terms “determine” or “determining” may include a variety of actions. For example, “determining” may include calculating, computing, processing, deriving, looking up (e.g., looking up in a table, a database or another data structure), ascertaining, or other actions. Also, “determining” may include receiving (e.g., receiving information or data), accessing (e.g., accessing data in a memory, data storage, distributed ledger, or over a network), or other actions. Also, “determining” may include resolving, selecting, choosing, establishing, or other similar actions.

As used herein, the terms “provide” or “providing” may include a variety of actions. For example, “providing” may include generating data, storing data in a location for later retrieval, transmitting data directly to a recipient, transmitting or storing a reference to data, or other actions. “Providing” may also include encoding, decoding, encrypting, decrypting, validating, verifying, or other actions.

As used herein, the term “access,” “accessing”, and other similar terms may include a variety of actions. For example, accessing data may include obtaining the data, examining the data, or retrieving the data. Providing access or providing data access may include providing confidentiality, integrity, or availability regarding the data.

As used herein, the term “message” may include one or more formats for communicating (e.g., transmitting or receiving) information or data. A message may include a machine-readable collection of information such as an Extensible Markup Language (XML) document, fixed-field message, comma-separated message, or another format. A message may, in some implementations, include a signal utilized to transmit one or more representations of information or data.

As used herein, the term “user interface” (also referred to as an interactive user interface, a graphical user interface or a UI), may refer to a computer-provided interface including data fields or other controls for receiving input signals or providing electronic information or for providing information to a user in response to received input signals. A user interface may be implemented, in whole or in part, using technologies such as hyper-text mark-up language (HTML), a programming language, web services, or rich site summary (RSS). In some implementations, a user interface may be included in a stand-alone client software application configured to communicate in accordance with one or more of the aspects described.

As used herein, the term “modify” or “modifying” may include several actions. For example, modifying data may include adding additional data or changing the already-existing data. As used herein, the term “obtain” or “obtaining” may also include several types of action. For example, obtaining data may include receiving data, generating data, designating data as a logical object, or other actions.

As used herein, the term “data object” may include a logical container for data. A data object may include an instance of an object in a software application implemented with an object-oriented programming language. A data object may include data formatted in an electronic data interchange (EDI) format, such as an eXtensible Markup Language (XML) object, a JavaScript Object Notation (JSON) object, or some other EDI-formatted object. A data object may include one or more functions that may manipulate the data of the data object. For example, a data object may include the functions or methods of an object in a software application implemented with an object-oriented programming language.

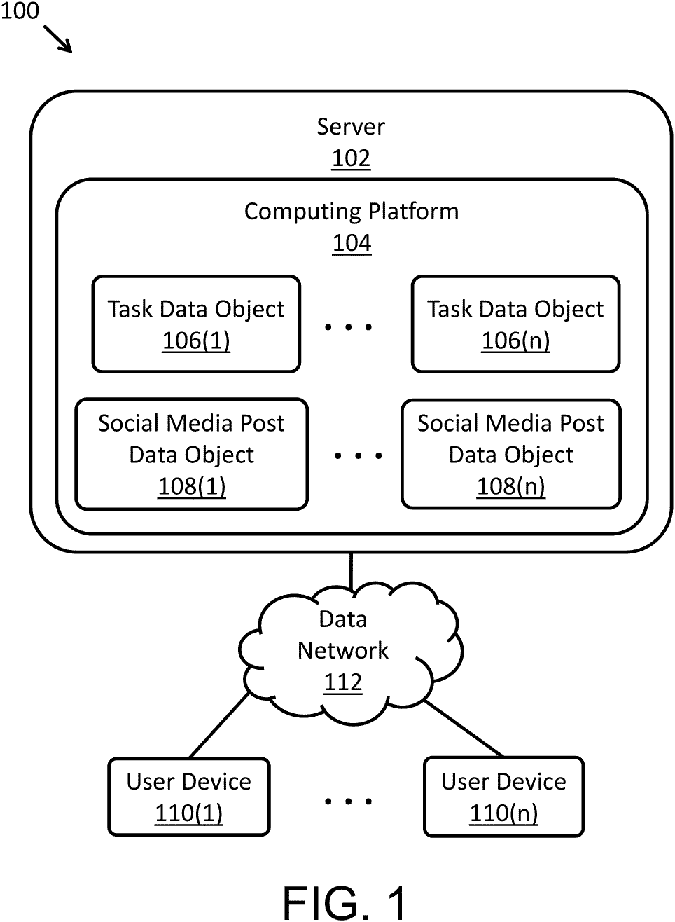

FIG. 1 depicts one embodiment of a system 100. The system 100 may include a system for a treatment and social media platform. The system 100 may include a server 102. The server 102 may include a computing platform 104. The computing platform 104 may include one or more task data objects 106(1)-(n). The computing platform 104 may include one or more social media post data objects 108(1)-(n). The system 100 may include one or more user devices 110(1)-(n). The system 100 may include a data network 112 over which various elements of the system 100 may be in data communication with each other.

As an overview, in some embodiments, a first user of the computing platform 104 may set a goal. The goal may include a task such as making positive statements about the user, identifying a strength of the user, meditating for a certain amount of time, or another task. The first user may use his or her user device 110(1) to create a first data packet containing data about the goal and send the first data packet to the computing platform 104. The computing platform 104 may create a task data object 106(1) based on the first data packet. The computing platform 104 may create a social media post on behalf of the first user about the goal. Other users of the computing platform 104 may be able to view the social media post on their respective user devices 110(2)-(n) or interact with the social media post.

Later, the first user may complete the goal and may use the user device 110(1) to create and send a second data packet to the computing platform 104 containing data about completing the goal (e.g., a completion date). The computing platform 104 may update the task data object 106(1) based on the second data packet. The computing platform 104 may update the first social media post to reflect that the first user has completed his/her goal, or the computing platform 104 may create another social media post on behalf of the first user about completing the goal. Other users of the computing platform 104 may be able to view the updated first social media post or the second social media post on their respective user devices 110(2)-(n) or interact with the social media post.

In some embodiments, the system 100 may allow a user of the computing platform 104 to track progress regarding a goal while combining such functionality with a scheduler and social media functionality. The ability of the users of the computing platform 104 to virtually encourage a user to complete his or her goals may help motivate the user and may increase the effectiveness of medical treatment.

Further details regarding the system 100 are now discussed. In one embodiment, the server 102 may include a computing device. The server 102 may include a processor. The server 102 may include a computer-readable storage medium. The computer-readable storage medium may include executable instructions stored therein. The processor, in response to executing the executable instructions, may be operable to carry out one or more steps or functions.

In one embodiment, the computing platform 104 may include a computing platform. The computing platform 104 may include treatment software that may allow a user of the platform 104 to set goals, track progress toward completing those goals, operate a calendar associated with the goals, or other goal-related functionality. In some embodiments, the computing platform 104 may store one or more user accounts. A user account may include data associated with a user of the platform 104. The user account may include a username (which may include a pseudonym in order for the user to remain anonymous), user demographic data, user contact data, or other user data. In one or more embodiments, the computing platform 104 may include social media software that may allow a user of the platform 104 to create or share information with other users of the platform. Users of the computing platform 104 may be able to virtually connect with other users of the platform 104 to create social networks, create social media posts, interact with other users’ social media posts, or perform other social media functionality.

In some embodiments, a task data object 106 may include a data object. The task data object may include data associated with a goal of a user of the computing platform 104. The task data object 106 may include text data. The text data may include text related to the goal. The text data may include a title of the goal, a description of the goal, or other goal-related text data. The task data object 106 may include a data field for a completion date. The completion data may include a date or time at which the user achieved the goal associated with the task data object 106.

In one embodiment, a social media post data object 108 may include a data object. The social media post data object 108 may include data associated with a social media post. The social media post data object 108 may include text data, image data, video data, or audio data. The social media post data object 108 may include a link to data on the Internet. The social media post data object 108 may include data or functionality that may allow users of the computing platform 104 to interact with the social media post that corresponds to the social media post data object 108. Such interactions may include a user’s ability to “like” the social media post, reply to the social media post (e.g., posting a comment that replies to the social media post), or other social media interactions. In some embodiments, the social media post data object 108 or the platform 104 may include configuration data that may govern which users of the platform 104 can view or interact with the social media post corresponding to the social media post data object 108.

In some embodiments, a user device 110 may include a computing device. The user device 110 may include one or more client software applications that may allow the user of the user device 110 to interact with the computing platform 104. A client software application may include a web browser, mobile application, or other software application.

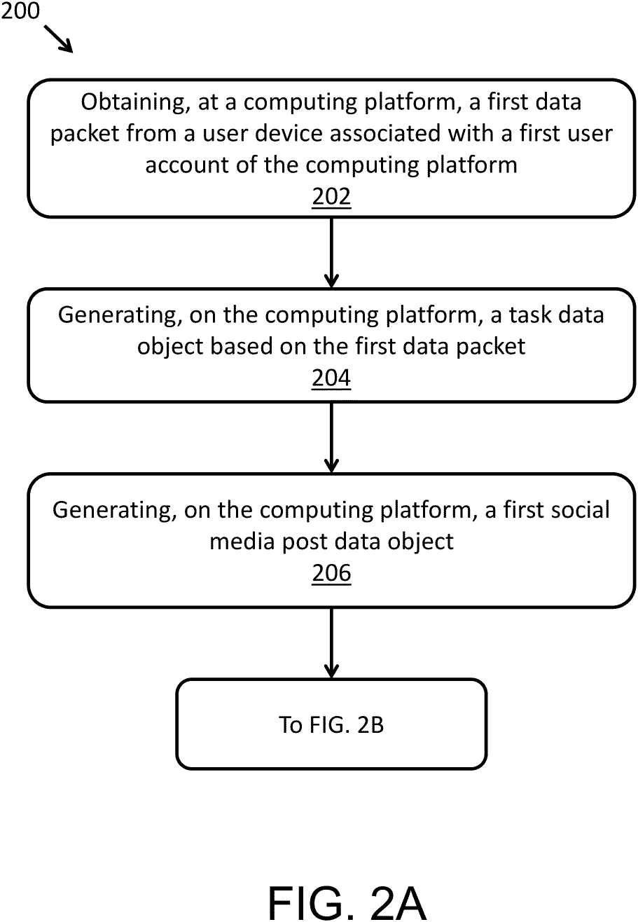

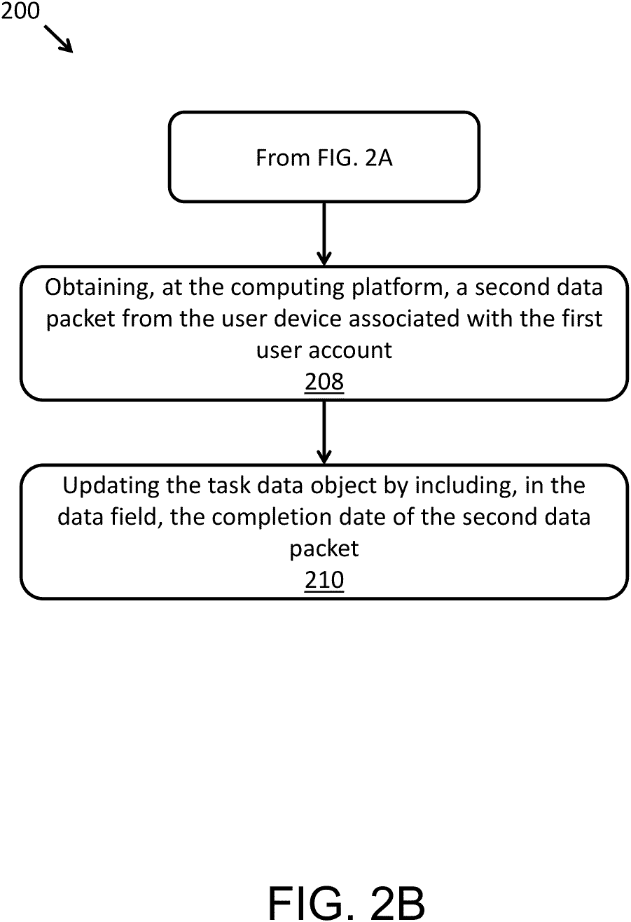

FIG. 2A and FIG. 2B depict one embodiment of a method 200. The method 200 may include a computer-implemented method. The method 200 may include a method for a treatment and social media platform. In some embodiments, the method 200 may include obtaining, at a computing platform 104, a first data packet from a user device 110(1) associated with a first user account of the computing platform 104 (step 202). The first data packet may include text data. The method 200 may include generating, on the computing platform 104, a task data object 106(1) based on the first data packet (step 204). The task data object 106(1) may include a data field for a completion date. The method 200 may include generating, on the computing platform 104, a first social media post data object 108(1) (step 206). The first social media post data object 108(1) may include at least a portion of the text data of the first data packet. The first social media post data object 108(1) may correspond to a first social media post, on the computing platform 104, that may be viewable by one or more users of the computing platform 104. The first social media post may include at least a portion the text data of the first data packet. The method 200 may include obtaining, at the computing platform 104, a second data packet from the user device 110(1) associated with the first user account (step 208). The second data packet may include a completion date. The method 200 may include updating the task data object 106(1) by including, in the data field, the completion date of the second data packet (step 210).

In one embodiment, obtaining the first data packet from the user device 110(1) associated with the first user account of the computing platform 104 (step 202) may include the user of the user device 110(1) using a UI of the user device 110(1) to enter data into the user device 110(1). The UI may include a UI of client software of the computing platform 104.

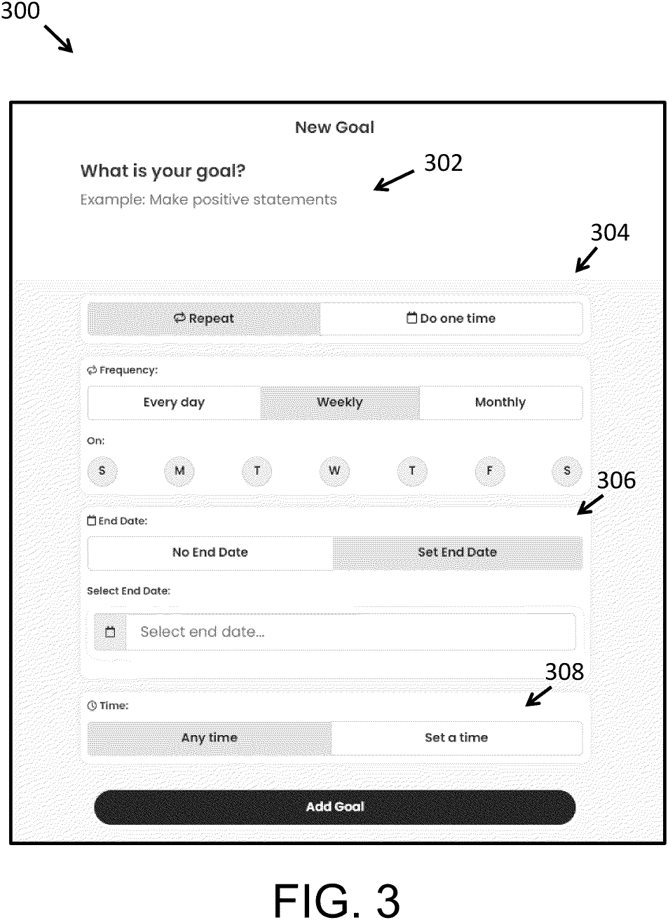

FIG. 3 depicts one example of a UI 300. The UI 300 may include a UI for entering data to create a goal on the computing platform 104 for a user of the platform 104. The UI 300 may include a goal text area 302. The goal text area 302 may include a text box where the user may input text data. The text data may include a title of the goal, further information about the goal, or other text data. The UI 300 may include a frequency area 304. The frequency area 304 may include one or more input elements where the user may select whether the goal repeats or not. If the goal does repeat, the frequency area 304 may include an input element where the user may select a frequency at which the goal repeats. The goal may repeat daily, weekly, monthly, or at some other interval. If the goal repeats weekly, the frequency area 304 may include a day of the week that the user can select. If the goal repeats monthly, the frequency area may include a day of the month that the user can select. The UI 300 may include an end date area 306. The end date area 306 may include an input element where the user may select whether the goal has an end date. If the goal has an end date, the end date area 306 may include an input area where the user may input or select an end date. The UI 300 may include a time area 308. The time area 308 may include an input element where the user may select whether the goal occurs at a certain time. If the goal occurs at a certain time, the time area 308 may include an input area where the user may input or select a time value.

The user device 110(1) may use the data entered via the UI 300 to create the first data packet. The first data packet may include at least one scheduled date. The scheduled date may include the end date. The scheduled date may include a single date occurrence (e.g., the user selected a one-time goal occurrence in the frequency area 304). In some embodiments, the single date occurrence may include a specific date (e.g., the user selected a specific end date in the end date area 306) or may include an open date (e.g., the user selected no end date in the end date area 306). In one embodiment, the scheduled date may include multiple date occurrences. The multiple date occurrences may include multiple specific dates (e.g., the user selected a weekly or monthly occurrence in the frequency area 304). The multiple specific dates may be separated by a predetermined time interval (e.g., weekly, monthly, etc.). In some embodiments, the multiple date occurrences may include open date occurrences (e.g., the goal repeats but at no set interval). The user device 110(1) may send the first data packet to the computing platform 104 via the data network 112.

In one embodiment, generating the task date object 106(1) based on the first data packet (step 204) may include the computing platform 104 creating the task data object 106(1) from the data contained within the first data packet. The computing platform 104 may store the task data object 106(1). The platform 104 may store the task data object 106(1) on the platform 104 or in a data storage external from the platform 104 that is in data communication with the platform 104. In one embodiment, the user of the user device 110(1) may be able to view a visual representation of the task data object 106(1) on the user’s user device 110(1).

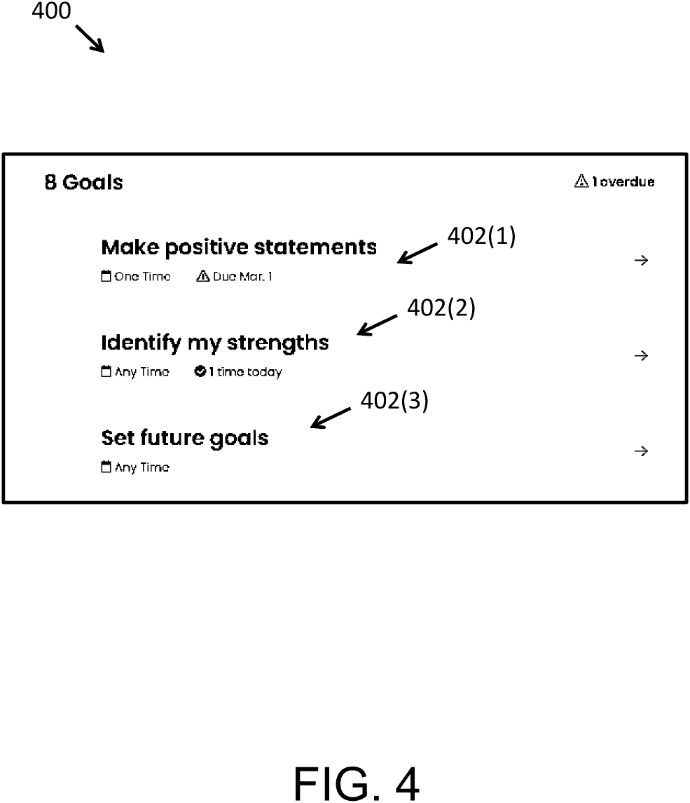

FIG. 4 depicts one embodiment of a UI 400. The UI 400 may include a UI for viewing or editing one or more goal data objects 106(1)-(n) of the user of the user device 110(1). The UI 400 may include one or more goal UI elements 402(1)-(n). A goal UI element 402 may include a visual data representation of a task data object 106. As can be seen in FIG. 4, a goal UI element 402 may include data from the task data object 106 such as the title of the goal, a frequency of the goal, an end date of the goal, or whether the goal has been completed. A user may interact with a goal UI element 402 in order for the UI 400 to display further information about the task data object 106 or in order to edit the task data object 106.

In one embodiment, generating, on the computing platform 104, the first social media post data object 108(1) (step 206) may include the computing platform 104 creating the first social media post data object 108(1) from the data contained within task data object 106(1). In one embodiment, the first social media post data object 108(1) may include at least a portion of the text data of the task data object 106(1) (which may be been based on the text data of the first data packet). The text data may include text related to the goal that corresponds to the task data object 106(1). The text data may include a title of the goal, a description of the goal, or other goal-related text data. The first social media post data object 108(1) may include a date of the task data object 106(1), such as whether the goal repeats, a frequency, and end date, or other date data discussed above in relation to the task data object 106(1).

In one or more embodiments, as discussed above, the computing platform 104 may include social media software. Generating, on the computing platform 104, the first social media post data object 108(1) (step 206) may include generating a social media post using the social media software of the platform 104. The social media post may correspond to the first social media post data object 108(1). The social media post may be viewable by one or more users of the computing platform 104. The social media post may be viewable by the one or more users via their respective user devices 110(1)-(n).

The social media post may include text data, image data, video data, audio data, a link to data on the Internet, or other data based on the data of the first social media post data object 108(1). For example, the social media post may include text data that may include the title of the goal represented by the corresponding task data object 106(1), or may include a description of the goal. The social media post may include a date of the task data object 106(1). The social media post may include an image that may be associated with the goal of the task data object 106(1) or an image that may draw attention to the social media post.

In some embodiments, the first user (i.e., the user who may have created the task data object 106(1) on the computing platform 104 and on whose behalf the platform 104 created the social media post) may configure the platform 104 or the social media post as to which users of the platform 104 can view the social media post. For example, the first user may configure the platform 104 such that only users that are “friends” or otherwise connected with the first user can view the social media post. In some embodiments, the first user may configure the visibility of a social media post on a social media post-by-post basis or on a general basis.

In certain embodiments, the computing platform 104 may obtain a third data packet from a user device associated with a second user account of the computing platform 104. The second user may be different from the first user (i.e., the user who may have created the task data object 106(1) on the computing platform 104 and on whose behalf the platform 104 created the social media post). The third data packet may include interaction data associated with the first social media post. The interaction data may include a social media reaction. A social media reaction may include a “like,” “favorite,” “thumbs-up,” or other reaction that a user may provide regarding a social media post. The interaction data may include a comment. The comment may include text data written by the second user. The text data may include text data responding to the social media post (e.g., words of encouragement, advice, etc.). The comment may include other data such as image data, video data, audio data, a link to data on the Internet, or other data.

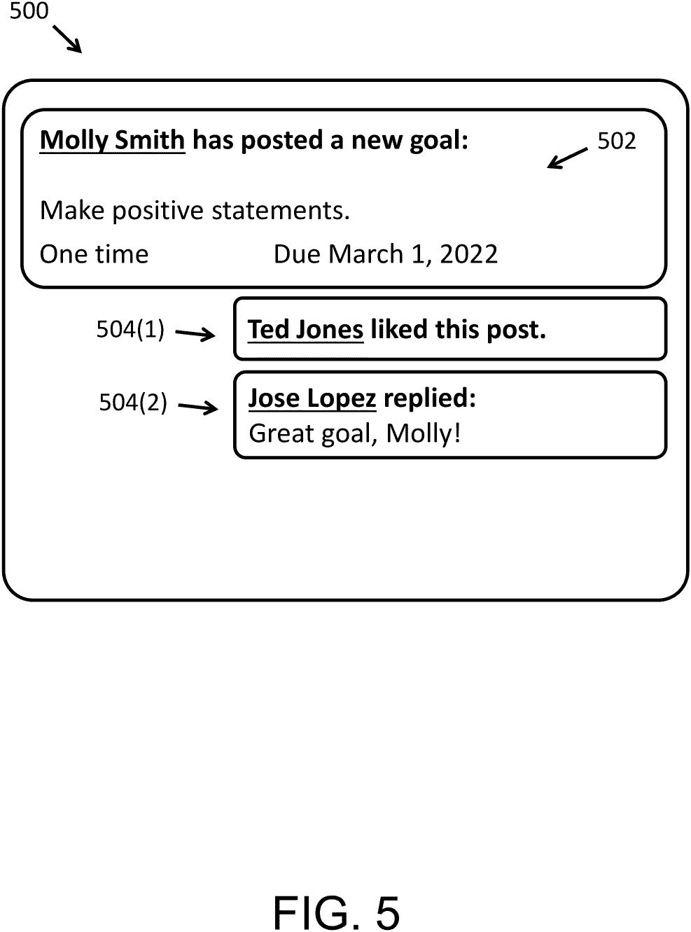

FIG. 5 depicts one embodiment of a UI 500. The UI 500 may display a social media post 502. As can be seen, the social media post 502 may include data indicating the first user (i.e., in the example of FIG. 5, “Molly Smith”), or that the first user has set a goal (“has posted a new goal”). The social media post 502 may include text data from the social media post data object 108(1) (which, in turn, may be based on the task data object 106(1)). The text data in the example social media post 502 may include “Make positive statements.” The social media post 502 may include date data (e.g., in the social media post 502 of FIG. 5, “One time” (indicating the goal does not repeat) and “Due Mar. 1, 2022” (indicating that the goal has an end date of Mar. 1, 2022).

The UI 500 may also display data from interaction data 504 associated with the social media post 502. The interaction data 504 may include a social media reaction 504(1). The interaction data 504 may include a comment 504(2).

In some embodiments, obtaining, at the computing platform 104, a second data packet from the user device 110(1) associated with the first user account (step 208) may include may include the user of the user device 110(1) using a UI of the user device 110(1) to enter data into the user device 110(1). The UI may include a UI of client software of the computing platform 104. The data may indicate that the first user has completed the goal associated with the task data object 106(1). The data may include a completion date (e.g., a calendar date or time), a difficult rating (e.g., a value indicating how difficult it was for the first user to complete the goal), text data (e.g., notes or other text), or other data. The user device 110(1) may use the data provided from the UI to generate the second data packet and send it to the computing platform 104.

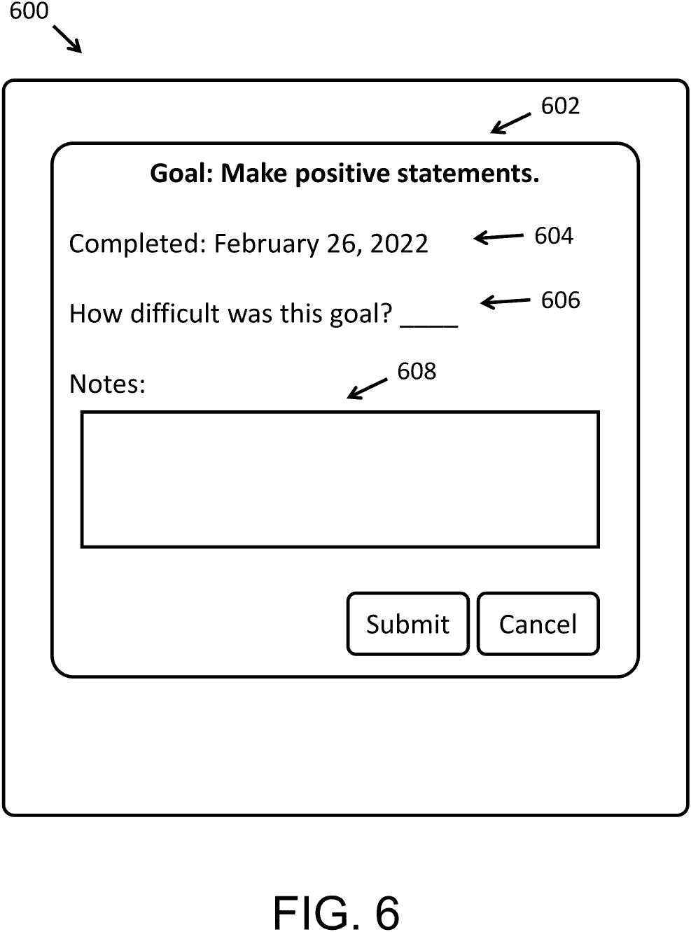

FIG. 6 depicts one embodiment of a UI 600. The UI 600 may include a goal screen 602. The goal screen 602 may include one or more UI elements by which the first user may enter information about completing the goal so that the user device 110(1) can use the information to generate the second data packet. In some embodiments, the user device 110(1) may display the UI 600 or the goal screen 602 in response to the first user interacting with a goal UI element 402 of the UI 400 of the FIG. 4.

In one embodiment, the goal screen 602 may include a completion date area 604. The completion date area 604 may include a UI element that the first user can use to enter a completion date or time. The completion date area 604 may include a text box, calendar UI element, scroll wheel, or other UI element that the first user can use to provide the completion date or time. In some embodiments, the completion date area 604 may automatically detect the current date or time from the user device 110(1). The goal screen 602 may include a difficulty area 606. The difficulty area 606 may include a UI element that the first user can use to enter a difficulty that the user experienced in completing the goal. The difficulty area 606 may include a text box, scroll wheel, slider, or other UI element. The goal screen 608 may include a notes area 608. The notes area 608 may include a text box or other UI element that the first user may use to include notes or other data in the second data packet.

In some embodiments, updating the task data object 106(1) (step 210) may include the computing platform 104 receiving the second data packet. The platform 104 may extract at least a portion of the data from the second data packet and use the extracted data to update the task data object 106(1). For example, the platform 104 may extract the completion date from the second data packet and may include, in a completion date data field of the task data object 106(1), the extracted completion date. The platform 104 may update the task data object 106(1) with a difficulty rating or notes extracted from the second data packet.

In one embodiment, in response to updating the task data object 106(1) (step 210), the computing platform 104 may update the social media post data object 108(1) with the completion date of the updated task data object 106(1). The platform 104 may also update the first social media post with the completion date. In some embodiments, the platform 104 may modify the social media post in other ways to highlight the fact that the social media post has updated in response to the first user completing the goal. Other ways of modifying the social media post may include changing the color of the social media post, including a distinctive border, or other modifications.

In some embodiments, in response to updating the task data object 106(1) (step 210), the computing platform 104 may generate, on the computing platform 104, a second social media post data object 108(2). The second social media post data object 108(2) may include the completion date from the updated task data object 106(1). The platform 104 may generate a second social media post corresponding to the second social media post data object 108(2). The second social media post may be viewable by one or more users of the computing platform 104. The second social media post may be similar to the first social media post except that it may include a completion date.

By updating the first social media post or generating the second social media post, users of the platform 104 that are able to view the social media post can know that the first user has completed his/her goal. The users of the platform 104 may interact with the social media post (e.g., by providing a social media reaction 504(1) or providing a comment 504(2)) in order to encourage the first user, which may lead to more effective treatment.

In some embodiments, the treatment features of the computing platform 104 (e.g., the hardware or software related to setting goals, tracking goals, scheduling goals, etc.) may be hosted on a treatment platform, and the social media features of the computing platform 104 (e.g., the hardware or software related to social media functionality) may be hosted on a social media platform. The treatment platform and the social media platform may be hosted on different servers. The treatment platform and the social media platform may be owned, operated, or controlled by different entities.

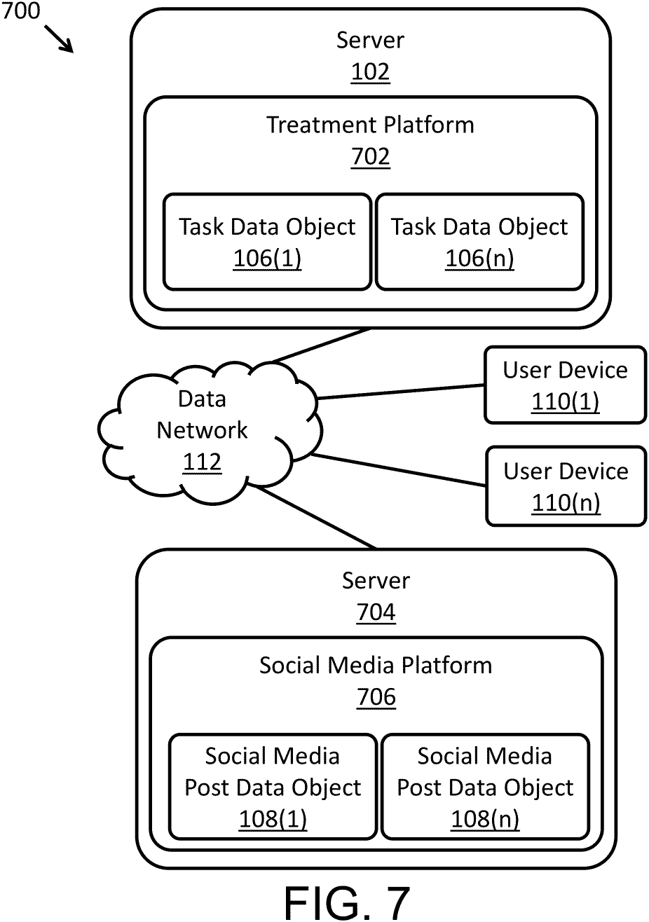

FIG. 7 depicts one embodiment of a system 700. The system 700 may include one or more elements of the system 100 of FIG. 1 such as the server 102, the task data objects 106(1)-(n), the social media post data objects 108(1)-(n), the user devices 110(1)-(n), or the data network 112. The system 700 may include a treatment platform 702. The system 700 may include a second server 704. The second server 704 may include a social media platform 706. As can be seen from FIG. 7, the treatment platform 702 and the social media platform 706 may be hosted on different servers 102, 704.

In some embodiments, the treatment platform 702 may include the functionality of the computing platform 104 related to setting goals, tracking goals, scheduling goals, etc. For example, the treatment platform 702 may carry out some of the functionality of the steps 202, 204, 208, or 210 of the method 200 of FIGS. 2A-B. The social media platform 706 may include the functionality of the computing platform 104 related to social media features. For example, the social media platform 706 may carry out some of the functionality of step 206 of the method 200. The social media platform 706 may carry out some of the functionality related to updating the social media post data object 108(1) or generating a second social media post data object 108(2) or updating the corresponding social media posts, as discussed above.

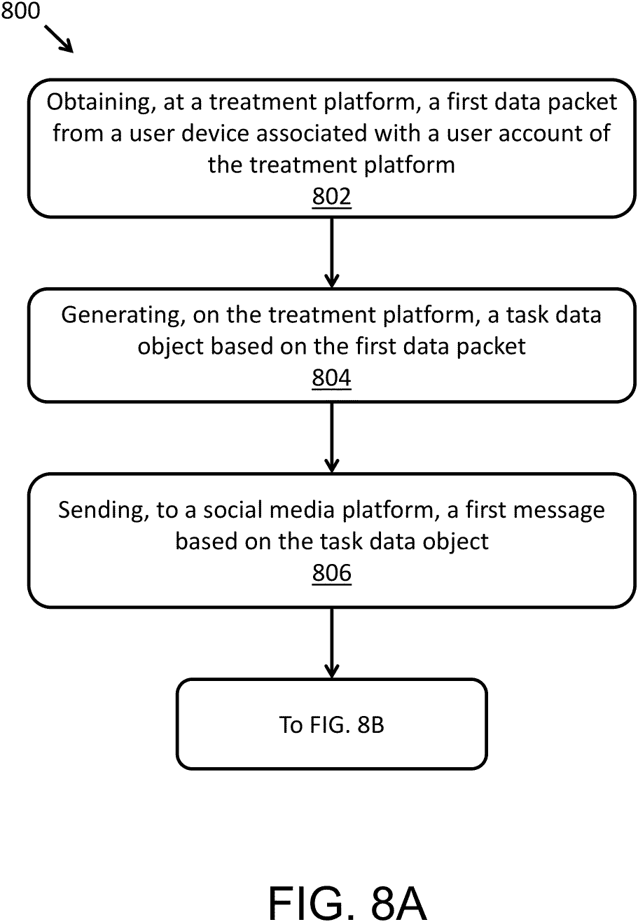

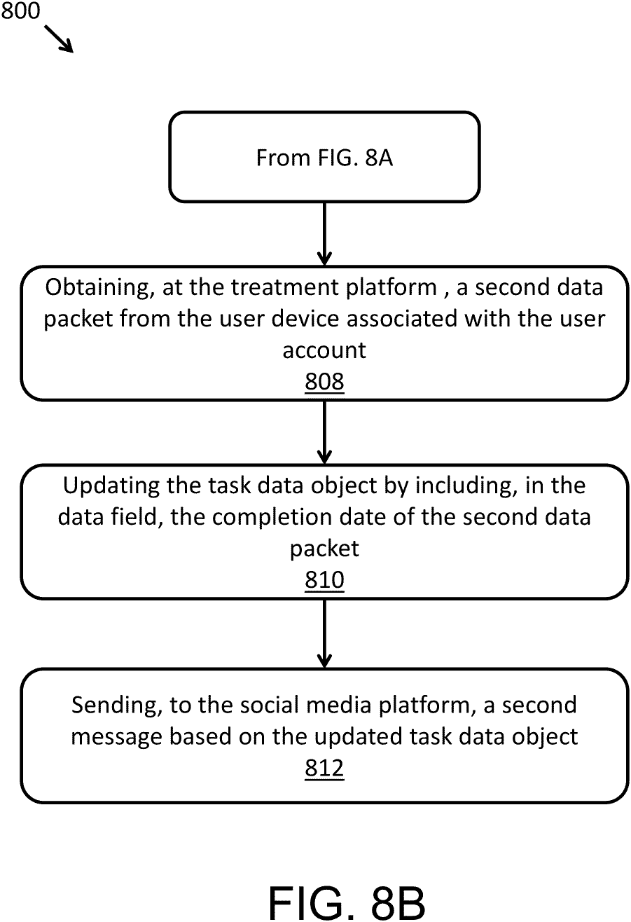

FIG. 8A and FIG. 8B depict one embodiment of a method 800. The method 800 may include a computer-implemented method for a treatment and social media platform. The method 800 may be carried out by one or more components of the system 700 of FIG. 7.

The method 800 may include obtaining, at a treatment platform 702, a first data packet from a user device 110(1) associated with a user account of the treatment platform 702 (step 802). The first data packet may include text data. The step 802 may be similar to the step 202 of the method 200 of FIGS. 2A-B. The method 800 may include generating, on the treatment platform 704, a task data object 106(1) based on the first data packet (step 804). The task data object 106(1) may include a data field for a completion date. The step 804 may be similar to the step 204 of the method 200.

The method 800 may include sending, to a social media platform 706, a first message based on the task data object 106(1) (step 806). The first message may include at least a portion the text data of the first data packet. The portion of the text data may be viewable by one or more users of the social media platform 706. For example, the social media platform 706 may generate a social media post data object 108(1) based on at least a portion of the data of the first message. The social media platform 706 may generate a corresponding social media post, and the social media post may be viewable by one or more users of the social media platform 706, as has been described above.

The method 800 may include obtaining, at the treatment platform 702, a second data packet from the user device 110(1) associated with the user account (step 808). The second data packet may include a completion date. The step 808 may be similar to the step 208 of the method 200. The method 800 may include updating the task data object 106(1) by including, in the data field, the completion date of the second data packet (step 810). The step 810 may be similar to the step 210 of the method 200.

The method 800 may include sending, to the social media platform 706, a second message based on the updated task data object 106(1) (step 812). The second message may include the completion date. The completion date may be viewable by one or more users of the social media platform 706. For example, the social media platform 706 may update the social media post data object 108(1) or may generate a second social media post data object 108(1) based on the data of the second message. The social media platform 706 may update the social media post or may generate a second social media post based on the updated social media post data object 108(1) or the second social media post data object 108(2), as has been discussed above.

While the making and using of various embodiments of the present disclosure are discussed in detail herein, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatuses, systems, and methods described herein. Such equivalents are considered to be within the scope of this disclosure and may be covered by the claims.

Furthermore, the described features, structures, or characteristics of the disclosure may be combined in any suitable manner in one or more embodiments. In the description contained herein, numerous specific details are provided, such as examples of programming, software, user selections, hardware, hardware circuits, hardware chips, or the like, to provide understanding of embodiments of the disclosure. One skilled in the relevant art will recognize, however, that the disclosure may be practiced without one or more of the specific details, or with other methods, components, materials, apparatuses, devices, systems, and so forth. In other instances, well-known structures, materials, or operations may not be shown or described in detail to avoid obscuring aspects of the disclosure.

These features and advantages of the embodiments will become more fully apparent from the description and appended claims, or may be learned by the practice of embodiments as set forth herein. As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer-readable media having program code embodied thereon.

In some embodiments, a module may be implemented as a hardware circuit comprising custom (very large-scale integration) VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer-readable media.

In some embodiments, a module may include a smart contract hosted on a blockchain. The functionality of the smart contract may be executed by a node (or peer) of the blockchain network. One or more inputs to the smart contract may be read or detected from one or more transactions stored on or referenced by the blockchain. The smart contract may output data based on the execution of the smart contract as one or more transactions to the blockchain. A smart contract may implement one or more methods or algorithms described herein.

The computer program product may include a computer-readable storage medium (or media) having computer-readable program instructions thereon for causing a processor to carry out aspects of the present disclosure. The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium may include a portable computer diskette, a random access memory (“RAM”), a read-only memory (“ROM”), an erasable programmable read-only memory (“EPROM” or Flash memory), a static random access memory (“SRAM”), a hard disk drive (“HDD”), a solid state drive, a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations or block diagrams of methods, apparatuses, systems, algorithms, or computer program products according to embodiments of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Thus, although there have been described particular embodiments of the present disclosure of a new and useful SYSTEMS AND METHODS FOR A TREATMENT AND SOCIAL MEDIA PLATFORM, it is not intended that such references be construed as limitations upon the scope of this disclosure.

What is claimed is:

1. A system, comprising:
a processor; and
a computer-readable storage medium with executable instructions stored therein, wherein the processor, in response to executing the executable instructions, is operable to:
obtain, at a computing platform, a first data packet from a user device associated with a first user account of the computing platform, wherein the first data packet includes text data,
generate, on the computing platform, a task data object based on the first data packet, wherein the task data object includes
at least a portion of the text data of the first data packet, and
a data field for a completion date,
generate, on the computing platform, a first social media post data object, wherein the first social media post data object
includes at least a portion of the text data of the first data packet, and
corresponds to a first social media post, on the computing platform, viewable by one or more users of the computing platform,
obtain, at the computing platform, a second data packet from the user device associated with the first user account, wherein the second data packet includes a completion date, and
update the task data object by including, in the data field, the completion date of the second data packet.

2. The system of claim 1, wherein the processor, in response to executing the executable instructions, is further operable to obtain, at the computing platform, a third data packet from a user device associated with a second user account of the computing platform, wherein:
the second user is different from the first user, and
the third data packet includes interaction data associated with the first social media post.

3. The system of claim 2, wherein the interaction data comprises at least one of:
a social media reaction; or
a comment.

4. The system of claim 1, wherein the processor, in response to executing the executable instructions, is further operable to:
update the first social media post data object with the completion date of the second data packet; and
update the first social media post with the completion date.

5. The system of claim 1, wherein the processor, in response to executing the executable instructions, is further operable to generate, on the computing platform, a second social media post data object, wherein the second social media post data object:
includes the completion date, and
corresponds to a second social media post, on the computing platform, viewable by one or more users of the computing platform.

6. The system of claim 5, wherein the processor, in response to executing the executable instructions, is further operable to obtain, at the computing platform, a third data packet from a user device associated with a second user account of the computing platform, wherein
the second user is different from the first user, and
the third data packet includes interaction data associated with the second social media post.

7. The system of claim 6, wherein the interaction data comprises at least one of:
a social media reaction; or
a comment.

8. A computer-implemented method for treatment, comprising:
obtaining, at a computing platform, a first data packet from a user device associated with a first user account of the computing platform, wherein the first data packet includes text data,
generating, on the computing platform, a task data object based on the first data packet, wherein the task data object includes
at least a portion of the text data of the first data packet, and
a data field for a completion date,
generating, on the computing platform, a first social media post data object, wherein the first social media post data object
includes at least a portion of the text data of the first data packet, and
corresponds to a first social media post, on the computing platform, viewable by one or more users of the computing platform,
obtaining, at the computing platform, a second data packet from the user device associated with the first user account, wherein the second data packet includes a completion date, and
updating the task data object by including, in the data field, the completion date of the second data packet.

9. The computer-implemented method of claim 8, wherein the first data packet further comprises at least one scheduled date.

10. The computer-implemented method of claim 9, wherein the at least one scheduled date comprises a single date occurrence.

11. The computer-implemented method of claim 10, wherein the single date occurrence includes a specific date.

12. The computer-implemented method of claim 10, wherein the single date occurrence includes an open date.

13. The computer-implemented method of claim 9, wherein the at least one scheduled date comprises a plurality of date occurrences.

14. The computer-implemented method of claim 13, wherein the plurality of date occurrences includes a plurality of specific dates.

15. The computer-implemented method of claim 14, wherein the plurality of specific dates comprises dates separated by a predetermined time interval.

16. The computer-implemented method of claim 13, wherein the plurality of date occurrences includes a plurality of open dates.

17. The computer-implemented method of claim 8, wherein the completion date includes a calendar date and a time.

18. A system, comprising:
a processor; and
a computer-readable storage medium with executable instructions stored therein, wherein the processor, in response to executing the executable instructions, is operable to:
obtain, at a treatment platform, a first data packet from a user device associated with a user account of the treatment platform, wherein the first data packet includes text data,
generate, on the treatment platform, a task data object based on the first data packet, wherein the task data object includes a data field for a completion date,
send, to a social media platform, a first message based on the task data object, wherein
the first message includes at least a portion the text data of the first data packet, and
the at least a portion of the text data is viewable by one or more users of the social media platform,
obtain, at the treatment platform, a second data packet from the user device associated with the user account, wherein the second data packet includes a completion date,
update the task data object by including, in the data field, the completion date of the second data packet, and
send, to the social media platform, a second message based on the updated task data object, wherein
the second message includes the completion date, and
the completion date is viewable by one or more users of the social media platform.
